(19) United States
(12) Patent Application Publication
(10) Pub. No.: US 2026/0162779 A1
(43) Pub. Date: Jun. 11, 2026

(54) METHODS FOR MONITORING FABRICATION OF WORKPIECES, ANALYTICAL COMPUTING DEVICES AND METHODS FOR TRAINING NEURAL NETWORKS TO ANALYZE ROBOT-ASSISTED DRILLING OPERATIONS

(71) Applicant: The Boeing Company, Arlington, VA (US)

(72) Inventors: Seth Yakel, Seattle, WA (US); Andreas Bartl, Munich (DE); Jose A. Blanco, Madrid (ES); Tobias Weber, Augsburg (DE); Victor Singh, Seattle, WA (US); Kwok Tung Chan, Mercer Island, WA (US); Mahesh Chengalva, Bothell, WA (US); Donald J. Best, III, Stanwood, WA (US)

(73) Assignee: The Boeing Company, Arlington, VA (US)

(21) Appl. No.: 18/976,804

(22) Filed: Dec. 11, 2024

(51) Int. Cl. G16C 60/00; B23B 49/00

(52) U.S. Cl. CPC G16C 60/00; B23B 49/00; B23B 2270/483

(57) ABSTRACT

Methods for monitoring fabrication of a workpiece includes receiving a thrust force signal from a robotic manipulator during a robot-assisted drilling operation on the workpiece, filtering noise data points and filtering outlier data points from the thrust force signal to form a filtered thrust force signal, extracting a thrust feature from the filtered thrust force signal, processing the thrust feature using a neural network trained to analyze the robot-assisted drilling operation and predicting a stiffness value for the workpiece based on a regression analysis of the thrust feature by the neural network during the processing of the one or more thrust feature. Analytical computing devices associated with the methods includes a network interface, a processor, an application program storage device, a neural network and a data storage device. Methods for training the neural network to analyze the robot-assisted drilling operation are also disclosed.

### FIELD

The present disclosure relates generally to monitoring robot-assisted drilling operations and, particularly, to monitoring such operations during fabrication of workpieces. The monitoring techniques using analytical computing devices with neural networks trained to analyze such drilling operations and predict parameters indicative of conditions of assembly that can lead to poor quality holes and damage to drilling tools. The neural network can also be trained to predict parameters indicative of missing fasteners in the workpiece. Methods for training the neural networks to analyze robotic-assisted drilling operations are also disclosed.

### BACKGROUND

During robot assisted drilling operations, for example, on an airplane wing, there are often issues with hole quality due to deflection in the workpiece. The deflection increases as the drill approaches further away from the baseline. The hole becomes oval due to the displacement, which causes the drill to wedge on the flanks as it retracts, causing damage to the tool. Problems encountered during assembly include spontaneous and non-reproducible tool failures during robot-assisted drilling. This leads to poor hole quality (e.g. hole offsets, oval holes, chatter, etc.), as well as high cost due to tool breakage and rework. Current drilling systems with process feedback capabilities provide insight on how good the process was with respect to a previously baselined process. For example, drilling process parameters are set during qualification test on flat coupons and then set in stone in process control documents. No adaptive process parameters are used in production based on system/workpiece reaction to external factors (e.g., thrust versus flexibility of the part). Ultimately, the condition of assembly is defined by engineering personnel and all checks are performed manually. Under this circumstances, we find many unfilled holes at the final assembly due to missing fasteners that require the use of cap seals to mitigate EME issues in operation.

Accordingly, those skilled in the art continue with research and development efforts to improve techniques for condition of assembly monitoring during drilling processes and use of machine learning.

### SUMMARY

Disclosed are examples of methods for monitoring fabrication of workpieces, analytical computing devices and methods for training neural networks to analyze robot-assisted drilling operations. The following is a non-exhaustive list of examples, which may or may not be claimed, of the subject matter according to the present disclosure.

In an example, the disclosed method for monitoring fabrication of a workpiece includes: (1) receiving a thrust force signal from a robotic manipulator during a robot-assisted drilling operation on the workpiece, the workpiece including a first material layer and a second material layer, the robot-assisted drilling operation including drilling a hole for a fastener through the first material layer and then through the second material layer; (2) filtering noise data points and filtering outlier data points from the thrust force signal to form a filtered thrust force signal; (3) extracting one or more thrust feature from the filtered thrust force signal; (4) processing the one or more thrust feature using a neural network trained to analyze the robot-assisted drilling operation; and (4) predicting a stiffness value for at least one of the first material layer and the second material layer of the workpiece based on a regression analysis of the one or more thrust feature by the neural network during the processing of the one or more thrust feature.

In an example, the disclosed analytical computing device includes a network interface, at least one processor and associated memory, at least one application program storage device, a neural network and at least one data storage device. The network interface in operative communication with a communication network. The at least one processor in operative communication with a robotic manipulator via the network interface and the communication network. The at least one application program storage device storing a drilling operation analysis application program. The neural network trained to analyze a data set associated with a robot-assisted drilling operation. The at least one data storage device configured to at least temporarily store the data set associated with the robot-assisted drilling operation. The at least one processor and the network interface are configured to receive a thrust force signal from the robotic manipulator via the communication network during the robot-assisted drilling operation on a workpiece. The workpiece includes a first material layer and a second material layer. The robot-assisted drilling operation includes drilling a hole for a fastener through the first material layer and then through the second material layer. The at least one processor and the associated memory, in conjunction with the at least one processor running the drilling operation analysis application program, are configured to filter noise data points and outlier data points from the thrust force signal to form a filtered thrust force signal, extract one or more thrust feature from the filtered thrust force signal and provide the one or more thrust feature to the neural network. The neural network is configured to process the one or more thrust feature, perform a regression analysis on the one or more thrust feature, predict a stiffness value for at least one of the first material layer and the second material layer of the workpiece based at least in part on the regression analysis and at least temporarily store the stiffness value in the at least one data storage device.

In an example, the disclosed method for training a neural network to analyze a robot-assisted drilling operation includes: (1) receiving a plurality of thrust force signal data sets from a training data repository, each thrust force signal data set based on a thrust force signal generated at a robotic manipulator during a corresponding a robot-assisted drilling operation on a workpiece, the workpiece including a first material layer and a second material layer, the robot-assisted drilling operation including drilling a hole for a fastener through the first material layer and then through the second material layer; (2) filtering noise data points and filtering outlier data points from each thrust force signal data set to form a plurality of filtered thrust force signal data sets; (3) extracting one or more thrust feature from each filtered thrust force signal data set to obtain a plurality of thrust feature data sets, each thrust feature data set including the one or more thrust feature; (4) providing the plurality of thrust feature data sets to the neural network; and (5) training the neural network to predict a stiffness value for at least one of the first material layer and the second material layer of the workpiece based on a regression analysis of the one or more thrust feature of the plurality of thrust feature data sets.

Other examples of the disclosed methods for monitoring fabrication of workpieces, analytical computing devices and methods for training neural networks to analyze robot-assisted drilling operations will become apparent from the following detailed description, the accompanying drawings and the appended claims.

### BRIEF DESCRIPTION OF THE DRAWINGS

FIG. 1 is a flow diagram of an example of a method for monitoring fabrication of a workpiece;

FIG. 2 is a flow diagram of an example the filtering of noise data points and the filtering of outlier data points from FIG. 1;

FIG. 3, in combination with FIG. 1, is a flow diagram of another example of a method for monitoring fabrication of a workpiece;

FIG. 4, in combination with FIG. 1, is a flow diagram of yet another example of a method for monitoring fabrication of a workpiece;

FIG. 5, in combination with FIG. 1, is a flow diagram of still another example of a method for monitoring fabrication of a workpiece;

FIG. 6, in combination with FIGS. 1 and 5, is a flow diagram of still yet another example of a method for monitoring fabrication of a workpiece;

FIG. 7, in combination with FIG. 1, is a flow diagram of another example of a method for monitoring fabrication of a workpiece;

FIG. 8 is a flow diagram of an example the filtering of noise data points and the filtering of outlier data points from FIG. 7;

FIG. 9, in combination with FIGS. 1 and 7, is a flow diagram of yet another example of a method for monitoring fabrication of a workpiece;

FIG. 10, in combination with FIGS. 1 and 7, is a flow diagram of still another example of a method for monitoring fabrication of a workpiece;

FIG. 11, in combination with FIGS. 1 and 7, is a flow diagram of still yet another example of a method for monitoring fabrication of a workpiece;

FIG. 12, in combination with FIGS. 1, 7 and 11, is a flow diagram of another example of a method for monitoring fabrication of a workpiece;

FIG. 13 is a functional block diagram of an example of an analytical computing device;

FIG. 14 is a functional perspective drawing of a robotic manipulator monitoring fabrication of a workpiece during a drilling operation;

FIG. 15 is a graph of a thrust force signal over time during a robot-assisted drilling operation on a workpiece;

FIG. 16 is a graph of a displacement signal over time during a robot-assisted drilling operation on a workpiece;

FIG. 17 is a flow diagram of an example of a method for training a neural network to analyze a robot-assisted drilling operation;

FIG. 18 is a flow diagram of an example the filtering of noise data points and the filtering of outlier data points from FIG. 17;

FIG. 19, in combination with FIG. 17, is a flow diagram of another example of a method for training a neural network to analyze a robot-assisted drilling operation;

FIG. 20, in combination with FIG. 17, is a flow diagram of yet another example of a method for training a neural network to analyze a robot-assisted drilling operation;

FIG. 21 is a flow diagram of an example the filtering of noise data points and the filtering of outlier data points from FIG. 20;

FIG. 22, in combination with FIGS. 17 and 20, is a flow diagram of still another example of a method for training a neural network to analyze a robot-assisted drilling operation;

FIG. 23 is a functional block diagram of an example of a training system;

FIG. 24 is a block diagram of aircraft production and service methodology that implements one or more of the examples of methods for monitoring fabrication of workpieces disclosed herein; and

FIG. 25 is a schematic illustration of an aircraft that incorporates components fabricated using one or more of the examples of methods for monitoring fabrication of workpieces disclosed herein.

### DETAILED DESCRIPTION

The various examples of methods 100, 300, 400, 500, 600, 700, 900, 1000, 1100, 1200 for monitoring fabrication of workpieces 1322 disclosed herein provide techniques for predicting stiffness values 1332 for the workpieces 1322. The various examples of analytical computing devices 1300 disclosed herein enable the methods 100, 300, 400, 500, 600, 700, 900, 1000, 1100, 1200 and the techniques for predicting 110 the stiffness values 1332 for the workpieces 1322. The various examples of methods 1700, 1900, 2000, 2200 for training neural networks 1316 to analyze robot-assisted drilling operations 1402 disclosed herein provide techniques for training 1710 the neural networks 1316 used in the methods 100, 300, 400, 500, 600, 700, 900, 1000, 1100, 1200 and the analytical computing devices 1300. Various examples of training systems 2300 disclosed herein provide techniques for the training 1710 of the neural networks 1316 and enable the methods 1700, 1900, 2000, 2200 for the training 1710.

This disclosure describes a system to correlate the workpiece 1322 deflection with the drill signal/frequency to determine the condition of the drill and the quality of the hole 1404. Test results show a good correlation between the frequencies and the workpiece 1322 measured dynamic behavior. The recorded force signals during the drilling tests revealed that due to static displacement of the work piece, shear forces are introduced into the drill, which resulted in poor hole quality and tool failure.

The methods 100, 300, 400, 500, 600, 700, 900, 1000, 1100, 1200 for monitoring fabrication of workpieces 1322 and the analytical computing devices 1300 monitor the conformance of assembly during robot-assisted drilling operations 1402 using process feedback/time-series and machine learning applications to account for flexibility of the workpiece 1322 in final assembly. The analytical computing devices 1300 may implement machine learning-based analysis by extracting a time-series signal, slicing the signal to look for specific features, running width of pecks against previously trained data, running a regression model, and obtaining stiffness values 1332 of the workpiece 1322 and/or drilling location of the workpiece 1322. The analytical computing devices 1300 may react accordingly to the results of the analysis, such as modifying drilling parameters or flagging a suspect hole 1404. The analytical computing devices 1300 may correlate static displacement of the workpiece 1322 to shear forces introduced into the drill, to determine locations where holes 1404 were drilled in relation to expected drilling locations to determine the condition/quality of the holes 1404.

A machine learning approach analyzes process time-series signals (e.g., thrust, bending moment, etc.) to infer the flexibility of the workpiece 1322 through the changes in signals at the drilling location and comparing the signals to the flexibility expected from an engineering definition. This allows checking condition of assembly conformance (i.e., surrounding unfilled holes 1404 which lead to EME issues) and using adaptive drilling processes to optimize the drilling process based on the workpiece 1322 deflection.

The disclosed techniques provide automation of conformance of assembly using process feedback through different signals and machine learning applications to account for flexibility of the actual workpiece 1322 in final assembly. This can alert non-conformances and help bridge qualification conditions with actual assembly conditions.

The disclosed methods 100, 300, 400, 500, 600, 700, 900, 1000, 1100, 1200 use any available data source for drilling operation 1402 (e.g., sensor toolholder, smart spindle, etc.) to provide process feedback signals (e.g., time-series) and run the following machine learning-based analysis: (1) extract time-series signal, (2) slice signal and look for specific features (e.g., peaks from peck drilling phases), (3) run width of pecks against previously trained data (e.g., relation of signal to width can be classified), (4) run multi-layer perceptron regression model (e.g., or other neural network model, depending on the type of data available), (5) get stiffness value 1332 of the workpiece 1322 and or drilling location of the workpiece 1322, and (6) react accordingly (e.g. modify drilling parameters, flag neighbor unfilled hole 1404, etc.).

Certain technical features automate analysis of drilling force sensor data. The physical problem has been already tested and proven: It is possible to see a change in the time-series signal from the drilling process data to get wider as the flexibility of the workpieces 1322 is increased (e.g., either by its geometry or by the location of the drilling operation 1402 with respect to the location the part is fixed). Therefore, the amount of actual data to train the machine learning models and subsequently define key performance indicators that vary based on the model details.

The methods 100, 300, 400, 500, 600, 700, 900, 1000, 1100, 1200 for monitoring fabrication of workpieces 1322 and the analytical computing devices 1300 disclosed herein provide many advantageous possibilities. For example, (1) flagging holes 1404 not being conformant to condition of assembly (e.g., adjacent holes 1404 not as planned due to missing fastener 1406), (2) feeding results information to ad-hoc dashboards for mandatory actions for operators (e.g., related to EME escapements mitigation), (3) if deflection detected is trespassing on any set threshold, drilling can be stopped to preserve cutter and avoid future hole rework, and (4) modifying drilling parameters to get good quality holes 1404 by reducing workpiece 1322 deflection (e.g. automatically reduce feeding).

The methods 100, 300, 400, 500, 600, 700, 900, 1000, 1100, 1200 for monitoring fabrication of workpieces 1322 and the analytical computing devices 1300 disclosed herein can be implemented in any production line where there is feedback during the drilling process. This feedback can come from many different sources of sensors (e.g., thrust, torque, acceleration, radial displacements, etc.). Suppliers of certain workpieces 1322 can provide a live-data stream from their process to an end-item manufacturer for the analysis. This can be accomplished, for example, using a cloud-based data warehouse to store the data.

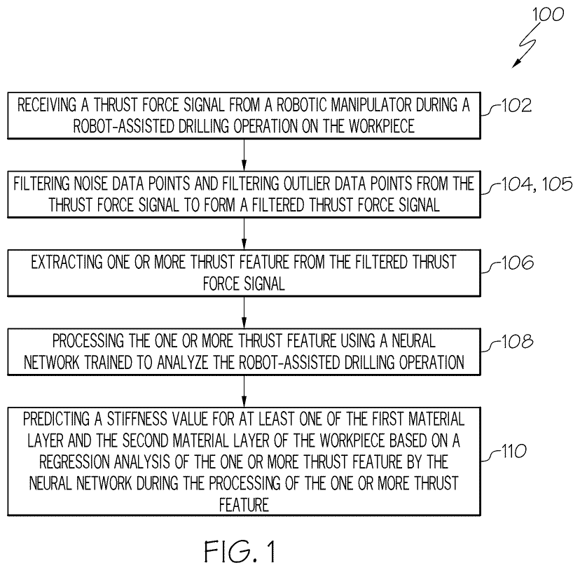

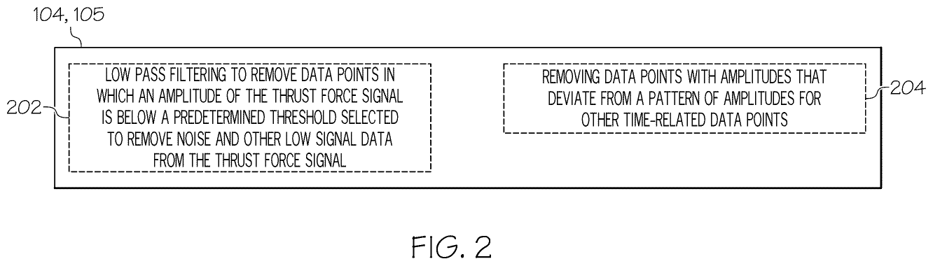

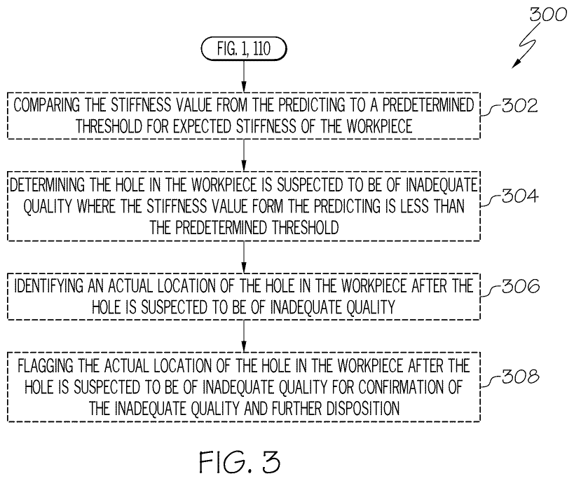

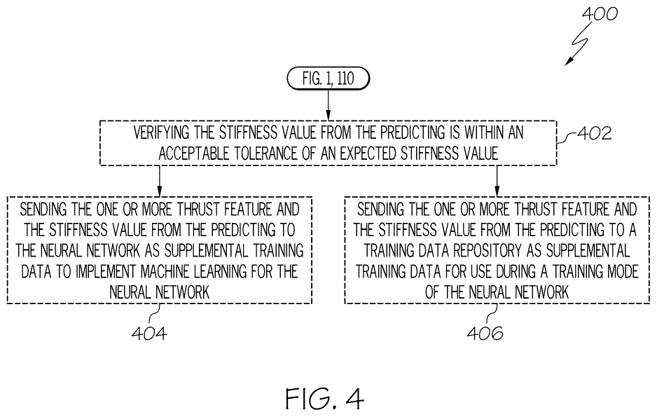

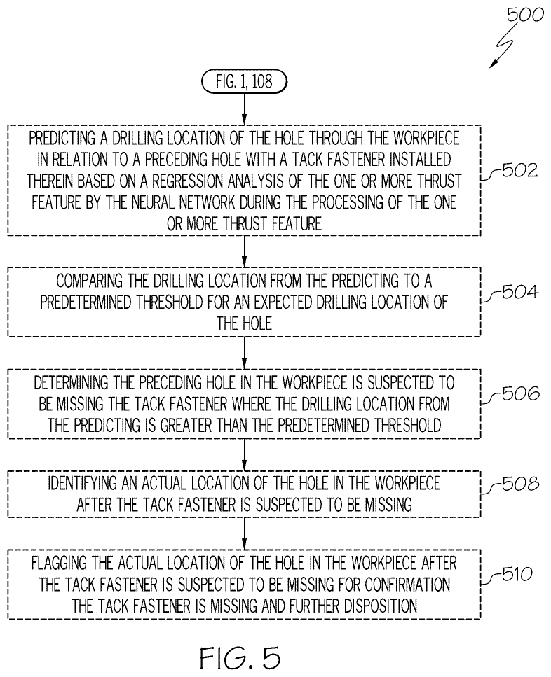

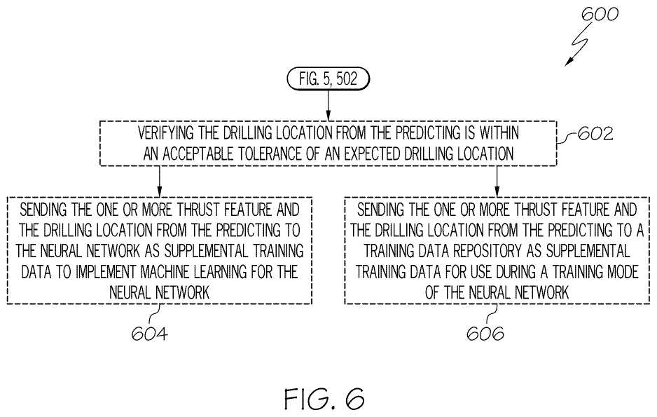

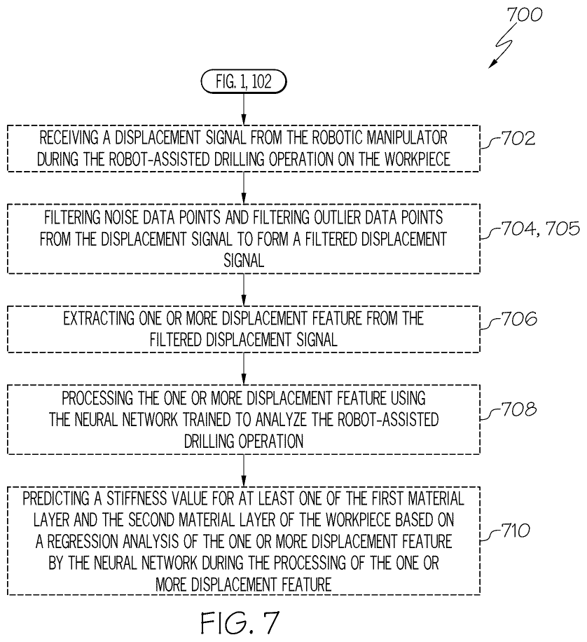

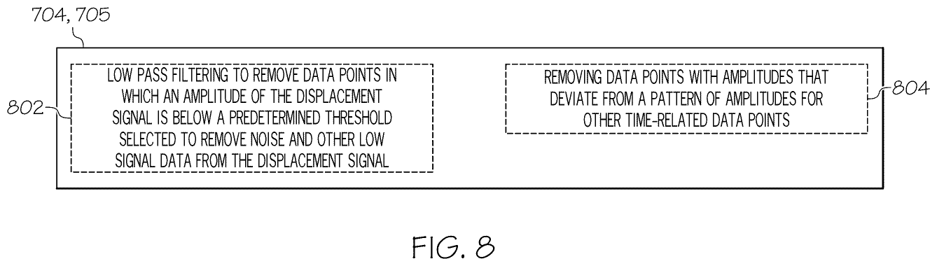

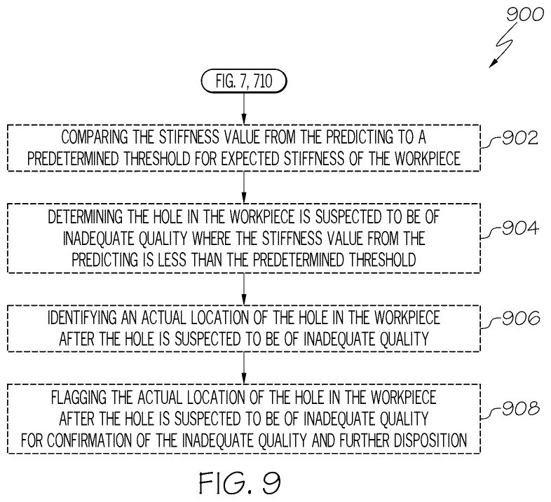

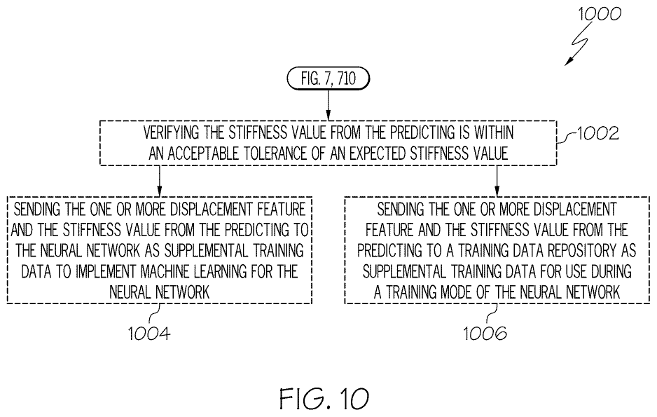

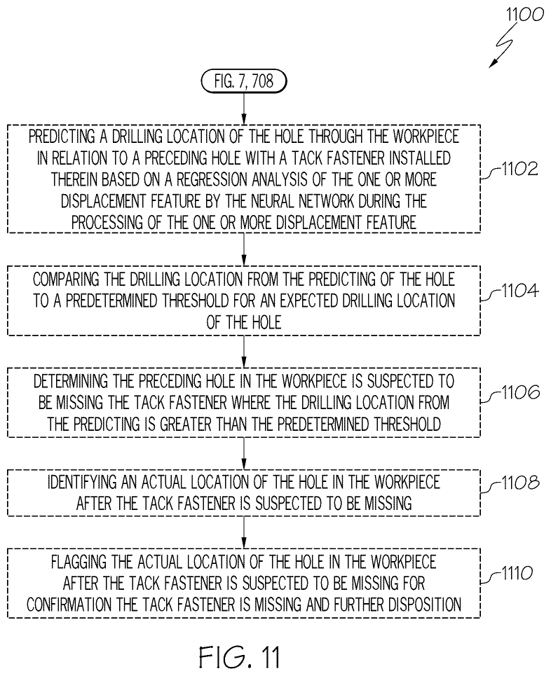

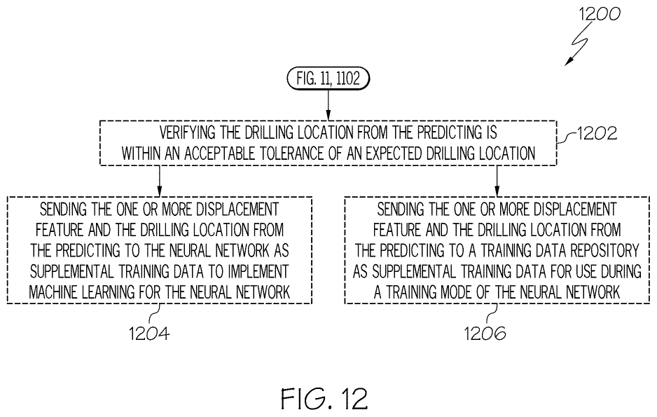

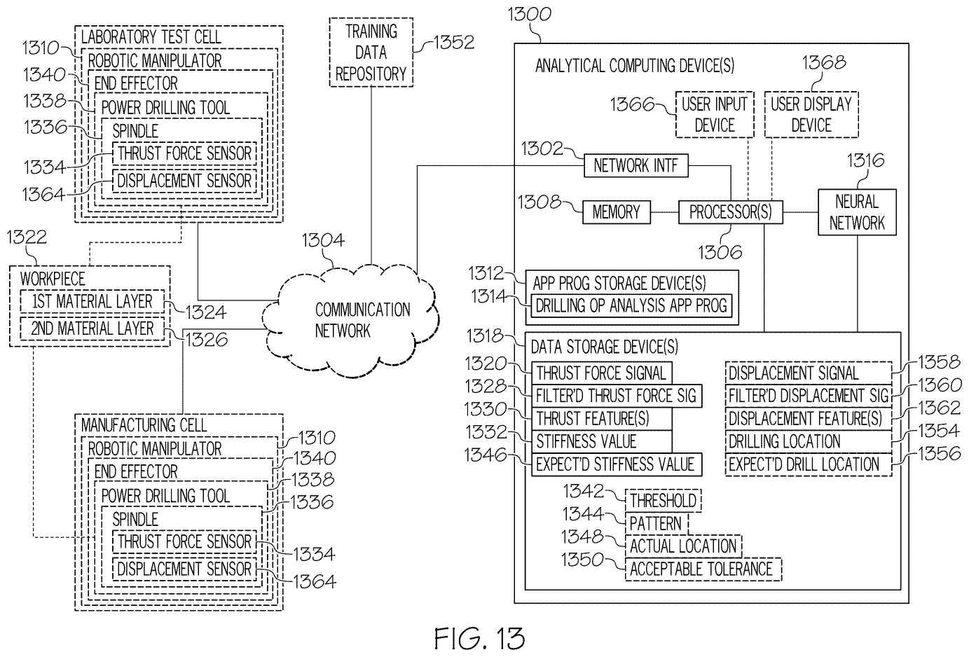

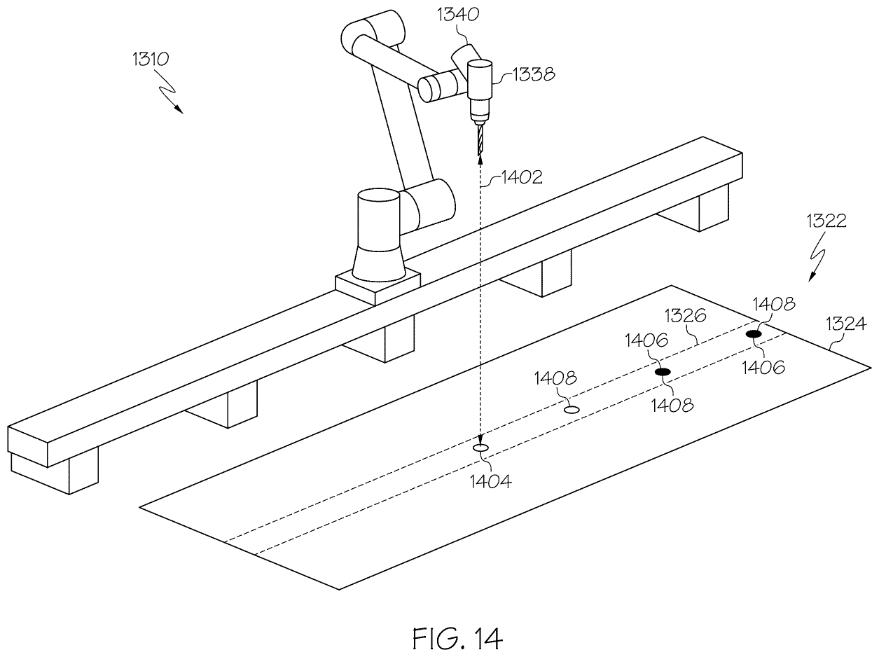

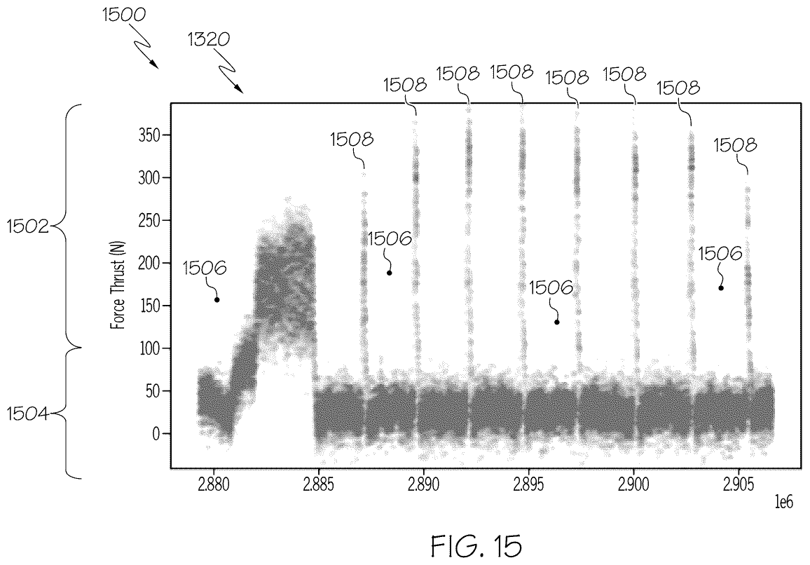

Referring generally to FIGS. 1-15, by way of examples, the present disclosure is directed to methods 100, 300, 400, 500, 600, 700, 900, 1000, 1100, 1200 for monitoring fabrication of workpieces 1322. FIG. 1 provides an example of the method 100 for monitoring fabrication of a workpiece. FIG. 2 provides an example the filtering 104 of noise data points and the filtering 105 of outlier data points from FIG. 1. FIG. 3, in combination with FIG. 1, provides an example of the method 300 for monitoring fabrication of a workpiece. FIG. 4, in combination with FIG. 1, provides an example of the method 400 for monitoring fabrication of a workpiece. FIG. 5, in combination with FIG. 1, provides an example of the method 500 for monitoring fabrication of a workpiece. FIG. 6, in combination with FIGS. 1 and 5, provides an example of the method 600 for monitoring fabrication of a workpiece. FIG. 7, in combination with FIG. 1, provides an example of the method 700 for monitoring fabrication of a workpiece. FIG. 8 provides an example the filtering 704 of noise data points and the filtering 705 of outlier data points from FIG. 7. FIG. 9, in combination with FIGS. 1 and 7, provides an example of the method 900 for monitoring fabrication of a workpiece. FIG. 10, in combination with FIGS. 1 and 7, provides an example of the method 1000 for monitoring fabrication of a workpiece. FIG. 11, in combination with FIGS. 1 and 7, provides an example of the method 1100 for monitoring fabrication of a workpiece. FIG. 12, in combination with FIGS. 1, 7 and 11, provides an example of the method 1200 for monitoring fabrication of a workpiece. FIG. 13 is a functional block diagram of an example of an analytical computing device 1300. FIG. 14 is a functional perspective drawing of a robotic manipulator 1310 monitoring fabrication of a workpiece 1322 during a drilling operation 1402. FIG. 15 is a graph 1500 of a thrust force signal 1320 over time during a robot-assisted drilling operation 1402 on a workpiece 1322.

With reference again to FIGS. 1, 2 and 13-15, in one or more example, a method 100 (see FIG. 1) for monitoring fabrication of a workpiece 1322 includes receiving 102 a thrust force signal 1320 from a robotic manipulator 1310 during a robot-assisted drilling operation 1402 on the workpiece 1322. The workpiece 1322 includes a first material layer 1324 and a second material layer 1326. The robot-assisted drilling operation 1402 includes drilling a hole 1404 for a fastener through the first material layer 1324 and then through the second material layer 1326. At 104, 105 noise data points 1504 and outlier data points 1506 are filtered from the thrust force signal 1320 to form a filtered thrust force signal 1328. At 106, one or more thrust feature 1330 is extracted from the filtered thrust force signal 1328. At 108, the one or more thrust feature 1330 is processed using a neural network 1316 trained to analyze the robot-assisted drilling operation 1402. At 110, a stiffness value 1332 for at least one of the first material layer 1324 and the second material layer 1326 of the workpiece 1322 is predicted based on a regression analysis of the one or more thrust feature 1330 by the neural network 1316 during the processing 108 of the one or more thrust feature 1330.

In another example of the method 100, the thrust force signal 1320 is generated by a thrust force sensor 1334 associated with a spindle 1336 of a power drilling tool 1338 on an end effector 1340 of the robotic manipulator 1310 during the robot-assisted drilling operation 1402. In yet another example of the method 100, the thrust force signal 1320 is indicative of a thrust force exerted upon the workpiece 1322 by the robotic manipulator 1310 over time during the robot-assisted drilling operation 1402. In still another example of the method 100, the robotic manipulator 1310 includes a robotic arm, an articulated robotic arm, a 4-axis robotic arm, a 6-axis robotic arm, a 7-axis robotic arm, a collaborative robot or any other suitable robotic manipulator in any suitable combination.

In still yet another example of the method 100, the first material layer 1324 of the workpiece 1322 includes a polymeric composite material, a thermoplastic composite material, a thermoset composite material, a metallic material, an aluminum material, an aluminum alloy material, a titanium material, a titanium alloy material or any other suitable material in any suitable combination. In another example of the method 100, the second material layer 1326 of the workpiece 1322 includes a metallic material, an aluminum material, an aluminum alloy material, a titanium material, a titanium alloy material, a polymeric composite material, a thermoplastic composite material, a thermoset composite material or any other suitable material in any suitable combination.

In yet another example of the method 100, the robot-assisted drilling operation 1402 also includes installing a tack fastener 1406 in the hole 1404 through the first material layer 1324 and the second material layer 1326. In still another example of the method 100, the filtering 104 of the noise data points 1504 includes low pass filtering 202 to remove data points in which an amplitude of the thrust force signal 1320 is below a predetermined threshold 1342 selected to remove noise and other low signal data from the thrust force signal 1320. In still yet another example of the method 100, the filtering 105 of the outlier data points 1506 includes removing 204 data points with amplitudes that deviate from a pattern 1344 of amplitudes for other time-related data points.

In another example of the method 100, the one or more thrust feature 1330 includes a width of each peck 1508 during the robot-assisted drilling operation 1402, an amount of pecks 1508 required to complete the robot-assisted drilling operation 1402, a thrust signal amplitude delta for each peck 1508 during the robot-assisted drilling operation 1402 and any other suitable thrust feature in any suitable combination. In yet another example of the method 100, the neural network 1316 includes a multi-layer perceptron model, a convolutional model, a recurrent model, a regression model, a classification model, a feedforward model, a back propagation model or any other suitable neural network model in any suitable combination.

With reference again to FIGS. 1, 3, 13 and 14, in one or more example, a method 300 (see FIG. 3) for monitoring fabrication of a workpiece 1322 includes the method 100 of FIG. 1 and continues from 110 to 302 where the stiffness value 1332 from the predicting 110 is compared to a predetermined threshold 1342 for an expected stiffness value 1346 for the workpiece 1322. At 304, the hole 1404 in the workpiece 1322 is determined to be suspected of inadequate quality where the stiffness value 1332 from the predicting 110 is less than the predetermined threshold 1342. At 306, an actual location 1348 of the hole 1404 in the workpiece 1322 is identified after the hole 1404 is suspected to be of inadequate quality. At 308, the actual location 1348 of the hole 1404 in the workpiece 1322 is flagged after the hole 1404 is suspected to be of inadequate quality for confirmation of the inadequate quality and further disposition.

With reference again to FIGS. 1, 4 and 13, in one or more example, a method 400 (see FIG. 4) for monitoring fabrication of a workpiece 1322 includes the method 100 of FIG. 1 and continues from 110 to 402 where the stiffness value 1332 from the predicting 110 is verified to be within an acceptable tolerance 1350 of an expected stiffness value 1346. In another example, the method 300 also includes sending 404 the one or more thrust feature 1330 and the stiffness value 1332 from the predicting 110 to the neural network 1316 as supplemental training data to implement machine learning for the neural network 1316. In yet another example, the method 300 also includes sending 406 the one or more thrust feature 1330 and the stiffness value 1332 from the predicting 110 to a training data repository 1352 as supplemental training data for use during a training mode of the neural network 1316.

With reference again to FIGS. 1, 5, 13 and 14, in one or more example, a method 500 (see FIG. 5) for monitoring fabrication of a workpiece 1322 includes the method 100 of FIG. 1 and continues from 108 to 502 where a drilling location 1354 of the hole 1404 through the workpiece 1322 is predicted in relation to a preceding hole 1408 with a tack fastener 1406 installed therein based on a regression analysis of the one or more thrust feature 1330 by the neural network 1316 during the processing 108 of the one or more thrust feature 1330. In another example, the method 500 also includes comparing 504 the drilling location 1354 from the predicting 502 to a predetermined threshold 1342 for an expected drilling location 1356 of the hole 1404. At 506, it is determined that the preceding hole 1408 in the workpiece 1322 is suspected to be missing the tack fastener 1406 where the drilling location 1354 from the predicting 502 is greater than the predetermined threshold 1342. At 508, an actual location 1348 of the hole 1404 in the workpiece 1322 is identified after the tack fastener 1406 is suspected to be missing. At 510, the actual location 1348 of the hole 1404 in the workpiece 1322 is flagged after the tack fastener 1406 is suspected to be missing for confirmation the tack fastener 1406 is missing and further disposition.

With reference again to FIGS. 1, 5, 6, and 13, in one or more example, a method 600 (see FIG. 6) for monitoring fabrication of a workpiece 1322 includes the method 100 of FIG. 1, the method 500 of FIG. 5 and continues from 502 to 602 where the drilling location 1354 from the predicting 502 is verified to be within an acceptable tolerance 1350 of an expected drilling location 1356. In yet another example, the method 600 also includes sending 604 the one or more thrust feature 1330 and the drilling location 1354 from the predicting 502 to the neural network 1316 as supplemental training data to implement machine learning for the neural network 1316. In yet another example, the method 600 also includes sending 606 the one or more thrust feature 1330 and the drilling location 1354 from the predicting 502 to a training data repository 1352 as supplemental training data for use during a training mode of the neural network 1316.

With reference again to FIGS. 1, 7, 13, 14 and 16, in one or more example, a method 700 (see FIG. 7) for monitoring fabrication of a workpiece 1322 includes the method 100 of FIG. 1 and continues from 102 to 702 where a displacement signal 1358 is received from the robotic manipulator 1310 during the robot-assisted drilling operation 1402 on the workpiece 1322. At 704, noise data points 1604 are filtered and at 705 outlier data points 1606 are filtered from the displacement signal 1358 to form a filtered displacement signal 1360. At 706, one or more displacement feature 1362 is extracted from the filtered displacement signal 1360. At 708, the one or more displacement feature 1362 is processed using the neural network 1316 trained to analyze the robot-assisted drilling operation 1402. At 710, a stiffness value 1332 for at least one of the first material layer 1324 and the second material layer 1326 of the workpiece 1322 is predicted based on a regression analysis of the one or more displacement feature 1362 by the neural network 1316 during the processing 708 of the one or more displacement feature 1362.

In another example of the method 700, the displacement signal 1358 is generated by a displacement sensor 1364 associated with a spindle 1336 of a power drilling tool 1338 on an end effector 1340 of the robotic manipulator 1310 during the robot-assisted drilling operation 1402. In a further example, the displacement signal 1358 is indicative of movement of the power drilling tool 1338 toward the workpiece 1322 during the robot-assisted drilling operation 1402. In yet another example of the method 700, the filtering 704 of the noise data points 1604 includes low pass filtering 802 to remove data points in which an amplitude of the displacement signal 1358 is below a predetermined threshold 1342 selected to remove noise and other low signal data from the displacement signal 1358. In still another example of the method 700, the filtering 705 of the outlier data points 1606 includes removing 804 data points with amplitudes that deviate from a pattern 1344 of amplitudes for other time-related data points. In still yet another example of the method 700, the one or more displacement feature 1362 includes a width of each peck 1608 during the robot-assisted drilling operation 1402, an amount of pecks 1608 required to complete the robot-assisted drilling operation 1402, a displacement amplitude delta for each peck 1608 during the robot-assisted drilling operation 1402 or any other suitable displacement feature in any suitable combination.

With reference again to FIGS. 1, 7, 9, 13 and 14, in one or more example, a method 900 (see FIG. 9) for monitoring fabrication of a workpiece 1322 includes the method 100 of FIG. 1, the method 700 of FIG. 7 and continues from 710 to 902 where the stiffness value 1332 from the predicting 710 is compared to a predetermined threshold 1342 for an expected stiffness value 1346 for the workpiece 1322. At 904, it is determined that the hole 1404 in the workpiece 1322 is suspected to be of inadequate quality where the stiffness value 1332 from the predicting 710 is less than the predetermined threshold 1342. At 906, an actual location 1348 of the hole 1404 in the workpiece 1322 is identified after the hole 1404 is suspected to be of inadequate quality. At 908, the actual location 1348 of the hole 1404 in the workpiece 1322 is flagged after the hole 1404 is suspected to be of inadequate quality for confirmation of the inadequate quality and further disposition.

With reference again to FIGS. 1, 7, 10 and 13, in one or more example, a method 1000 (see FIG. 10) for monitoring fabrication of a workpiece 1322 includes the method 100 of FIG. 1, the method 700 of FIG. 7 and continues from 710 to 1002 where the stiffness value 1332 from the predicting 710 is verified to be within an acceptable tolerance 1350 of an expected stiffness value 1346. In another example, the method 1000 also includes sending 1004 the one or more displacement feature 1362 and the stiffness value 1332 from the predicting 710 to the neural network 1316 as supplemental training data to implement machine learning for the neural network 1316. In yet another example, the method 1000 also includes sending 1006 the one or more displacement feature 1362 and the stiffness value 1332 from the predicting 710 to a training data repository 1352 as supplemental training data for use during a training mode of the neural network 1316.

With reference again to FIGS. 1, 7, 11, 13 and 14, in one or more example, a method 1100 (see FIG. 11) for monitoring fabrication of a workpiece 1322 includes the method 100 of FIG. 1, the method 700 of FIG. 7 and continues from 708 to 1102 where a drilling location 1354 of the hole 1404 through the workpiece 1322 is predicted in relation to a preceding hole 1408 with a tack fastener 1406 installed therein based on a regression analysis of the one or more displacement feature 1362 by the neural network 1316 during the processing 708 of the one or more displacement feature 1362. In another example, the method 1100 also includes comparing 1104 the drilling location 1354 from the predicting 1102 of the hole 1404 to a predetermined threshold 1342 for an expected drilling location 1356 of the hole 1404. At 1106, it is determined that the preceding hole 1408 in the workpiece 1322 is suspected to be missing the tack fastener 1406 where the drilling location 1354 from the predicting 1102 is greater than the predetermined threshold 1342. At 1108, an actual location 1348 of the hole 1404 in the workpiece 1322 is identified after the tack fastener 1406 is suspected to be missing. At 1110, the actual location 1348 of the hole 1404 in the workpiece 1322 is flagged after the tack fastener 1406 is suspected to be missing for confirmation the tack fastener 1406 is missing and further disposition.

With reference again to FIGS. 1, 7 and 11-13, in one or more example, a method 1200 (see FIG. 12) for monitoring fabrication of a workpiece 1322 includes the method 100 of FIG. 1, the method 700 of FIG. 7, the method 1100 of FIG. 11 and continues from 1102 to 1202 where the drilling location 1354 from the predicting 1102 is verified to be within an acceptable tolerance 1350 of an expected drilling location 1356. In another example, the method 1200 also includes sending 1204 the one or more displacement feature 1362 and the drilling location 1354 from the predicting 1102 to the neural network 1316 as supplemental training data to implement machine learning for the neural network 1316. In yet another example, the method 1200 also includes sending 1206 the one or more displacement feature 1362 and the drilling location 1354 from the predicting 1102 to a training data repository 1352 as supplemental training data for use during a training mode of the neural network 1316.

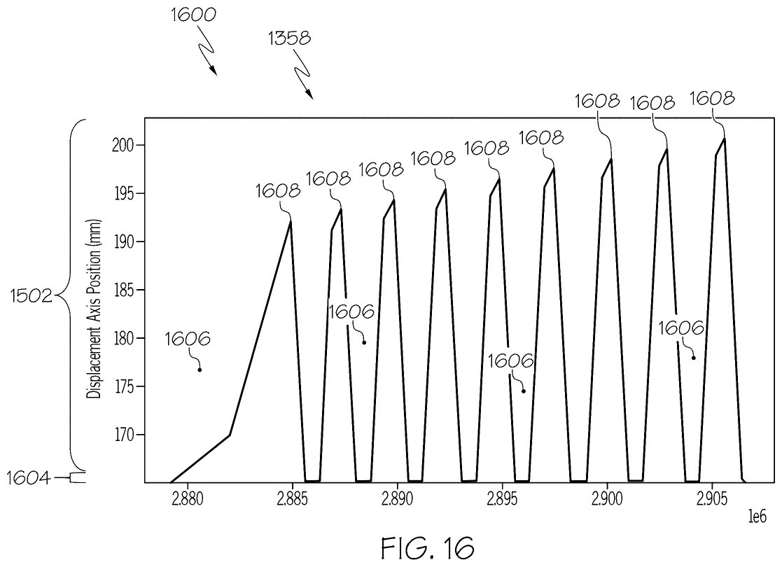

Referring generally to FIGS. 13-16, by way of examples, the present disclosure is directed to an analytical computing device 1300. FIG. 13 is a functional block diagram of an example of the analytical computing device 1300. FIG. 14 is a functional perspective drawing of a robotic manipulator 1310 monitoring fabrication of a workpiece 1322 during a drilling operation 1402. FIG. 15 is a graph 1500 of a thrust force signal 1320 over time during a robot-assisted drilling operation 1402 on a workpiece 1322. FIG. 16 is a graph 1600 of a displacement signal 1358 over time during a robot-assisted drilling operation 1402 on a workpiece 1322.

With reference again to FIGS. 13-16, in one or more example, an analytical computing device 1300 includes a network interface 1302, at least one processor 1306 and associated memory 1308, at least one application program storage device 1312, a neural network 1316 and at least one data storage device 1318. The network interface 1302 in operative communication with a communication network 1304. The at least one processor 1306 in operative communication with a robotic manipulator 1310 via the network interface 1302 and the communication network 1304. The at least one application program storage device 1312 storing a drilling operation analysis application program 1314. The neural network 1316 trained to analyze a data set 1502 associated with a robot-assisted drilling operation 1402. The at least one data storage device 1318 configured to at least temporarily store the data set 1502 associated with the robot-assisted drilling operation 1402. The at least one processor 1306 and the network interface 1302 are configured to receive a thrust force signal 1320 from the robotic manipulator 1310 via the communication network 1304 during the robot-assisted drilling operation 1402 on a workpiece 1322. The workpiece 1322 includes a first material layer 1324 and a second material layer 1326. The robot-assisted drilling operation 1402 includes drilling a hole 1404 for a fastener through the first material layer 1324 and then through the second material layer 1326. The at least one processor 1306 and the associated memory 1308, in conjunction with the at least one processor 1306 running the drilling operation analysis application program 1314, are configured to filter noise data points 1504 and outlier data points 1506 from the thrust force signal 1320 to form a filtered thrust force signal 1328, extract one or more thrust feature 1330 from the filtered thrust force signal 1328 and provide the one or more thrust feature 1330 to the neural network 1316. The neural network 1316 is configured to process the one or more thrust feature 1330, perform a regression analysis on the one or more thrust feature 1330, predict a stiffness value 1332 for at least one of the first material layer 1324 and the second material layer 1326 of the workpiece 1322 based at least in part on the regression analysis and at least temporarily store the stiffness value 1332 in the at least one data storage device 1318.

In another example of the analytical computing device 1300, the thrust force signal 1320 is generated by a thrust force sensor 1334 associated with a spindle 1336 of a power drilling tool 1338 on an end effector 1340 of the robotic manipulator 1310 during the robot-assisted drilling operation 1402. In yet another example of the analytical computing device 1300, the robotic manipulator 1310 includes a robotic arm, an articulated robotic arm, a 4-axis robotic arm, a 6-axis robotic arm, a 7-axis robotic arm, a collaborative robot or any other suitable robotic manipulator in any suitable combination. In still another example of the analytical computing device 1300, the robot-assisted drilling operation 1402 also includes installing a tack fastener 1406 in the hole 1404 through the first material layer 1324 and the second material layer 1326.

In still yet another example of the analytical computing device 1300, the at least one processor 1306 and the associated memory 1308, in conjunction with the at least one processor 1306 running the drilling operation analysis application program 1314, are configured to low pass filter the thrust force signal 1320 to remove data points in which an amplitude of the thrust force signal 1320 is below a predetermined threshold 1342 selected to remove noise and other low signal data from the thrust force signal 1320. In another example of the analytical computing device 1300, the at least one processor 1306 and the associated memory 1308, in conjunction with the at least one processor 1306 running the drilling operation analysis application program 1314, are configured to remove data points with amplitudes that deviate from a pattern 1344 of amplitudes for other time-related data points.

In yet another example of the analytical computing device 1300, the neural network 1316 includes a multi-layer perceptron model, a convolutional model, a recurrent model, a regression model, a classification model, a feedforward model, a back propagation model or any other suitable neural network model in any suitable combination.

In still another example of the analytical computing device 1300, the at least one processor 1306 and the associated memory 1308, in conjunction with the at least one processor 1306 running the drilling operation analysis application program 1314, are configured to compare the stiffness value 1332 predicted by the neural network 1316 to a predetermined threshold 1342 for an expected stiffness value 1346 for the workpiece 1322, determine the hole 1404 in the workpiece 1322 is suspected to be of inadequate quality where the stiffness value 1332 predicted by the neural network 1316 is less than the predetermined threshold 1342, identify an actual location 1348 of the hole 1404 in the workpiece 1322 after the hole 1404 is suspected to be of inadequate quality and flag the actual location 1348 of the hole 1404 in the workpiece 1322 after the hole 1404 is suspected to be of inadequate quality for confirmation of the inadequate quality and further disposition.

In still yet another example of the analytical computing device 1300, the at least one processor 1306 and the associated memory 1308, in conjunction with the at least one processor 1306 running the drilling operation analysis application program 1314, are configured to verify the stiffness value 1332 predicted by the neural network 1316 is within an acceptable tolerance 1350 of an expected stiffness value 1346. In a further example, the at least one processor 1306 and the associated memory 1308, in conjunction with the at least one processor 1306 running the drilling operation analysis application program 1314, are configured to send the one or more thrust feature 1330 and the stiffness value 1332 to the neural network 1316 as supplemental training data to implement machine learning for the neural network 1316. In another further example, the at least one processor 1306 and the network interface 1302, in conjunction with the at least one processor 1306 running the drilling operation analysis application program 1314, are configured to send the one or more thrust feature 1330 and the stiffness value 1332 predicted by the neural network 1316 to a training data repository 1352 as supplemental training data for use during a training mode of the neural network 1316.

In another example of the analytical computing device 1300, the neural network 1316 is configured to predict a drilling location 1354 of the hole 1404 through the workpiece 1322 in relation to a preceding hole 1408 with a tack fastener 1406 installed therein based on a regression analysis of the one or more thrust feature 1330 by the neural network 1316 during the processing of the one or more thrust feature 1330. In a further example, the at least one processor 1306 and the associated memory 1308, in conjunction with the at least one processor 1306 running the drilling operation analysis application program 1314, are configured to compare the drilling location 1354 predicted by the neural network 1316 to a predetermined threshold 1342 for an expected drilling location 1356 of the hole 1404, determine the preceding hole 1408 in the workpiece 1322 is suspected to be missing the tack fastener 1406 where the drilling location 1354 predicted by the neural network 1316 is greater than the predetermined threshold 1342, identify an actual location 1348 of the hole 1404 in the workpiece 1322 after the tack fastener 1406 is suspected to be missing and flag the actual location 1348 of the hole 1404 in the workpiece 1322 after the tack fastener 1406 is suspected to be missing for confirmation the tack fastener 1406 is missing and further disposition.

In another further example, the at least one processor 1306 and the associated memory 1308, in conjunction with the at least one processor 1306 running the drilling operation analysis application program 1314, are configured to verify the drilling location 1354 predicted by the neural network 1316 is within an acceptable tolerance 1350 of an expected drilling location 1356. In an even further example, the at least one processor 1306 and the associated memory 1308, in conjunction with the at least one processor 1306 running the drilling operation analysis application program 1314, are configured to send the one or more thrust feature 1330 and the drilling location 1354 to the neural network 1316 as supplemental training data to implement machine learning for the neural network 1316. In another even further example, the at least one processor 1306 and the associated memory 1308, in conjunction with the at least one processor 1306 running the drilling operation analysis application program 1314, are configured to send the one or more thrust feature 1330 and the drilling location 1354 predicted by the neural network 1316 to a training data repository 1352 as supplemental training data for use during a training mode of the neural network 1316.

In yet another example of the analytical computing device 1300, the at least one processor 1306 and the network interface 1302 are configured to receive a displacement signal 1358 from the robotic manipulator 1310 during the robot-assisted drilling operation 1402 on the workpiece 1322. In this example, the at least one processor 1306 and the associated memory 1308, in conjunction with the at least one processor 1306 running the drilling operation analysis application program 1314, are configured to filter noise data points 1604 and outlier data points 1606 from the displacement signal 1358 to form a filtered displacement signal 1360, extract one or more displacement feature 1362 from the filtered displacement signal 1360 and provide the one or more displacement feature 1362 to the neural network 1316. The neural network 1316 is configured to process the one or more displacement feature 1362 using the neural network 1316 trained to analyze the robot-assisted drilling operation 1402 and predict a stiffness value 1332 for at least one of the first material layer 1324 and the second material layer 1326 of the workpiece 1322 based on a regression analysis of the one or more displacement feature 1362 during the processing of the one or more displacement feature 1362.

In a further example, the displacement signal 1358 is generated by a displacement sensor 1364 associated with a spindle 1336 of a power drilling tool 1338 on an end effector 1340 of the robotic manipulator 1310 during the robot-assisted drilling operation 1402. In another further example, the at least one processor 1306 and the associated memory 1308, in conjunction with the at least one processor 1306 running the drilling operation analysis application program 1314, are configured to low pass filter the displacement signal 1358 to remove data points in which an amplitude of the displacement signal 1358 is below a predetermined threshold 1342 selected to remove noise and other low signal data from the displacement signal 1358. In yet another further example, the at least one processor 1306 and the associated memory 1308, in conjunction with the at least one processor 1306 running the drilling operation analysis application program 1314, are configured to remove data points with amplitudes that deviate from a pattern 1344 of amplitudes for other time-related data points.

In still another further example, the at least one processor 1306 and the associated memory 1308, in conjunction with the at least one processor 1306 running the drilling operation analysis application program 1314, are configured to compare the stiffness value 1332 predicted by the neural network 1316 to a predetermined threshold 1342 for an expected stiffness value 1346 for the workpiece 1322, determine the hole 1404 in the workpiece 1322 is suspected to be of inadequate quality where the stiffness value 1332 predicted by the neural network 1316 is less than the predetermined threshold 1342, identify an actual location 1348 of the hole 1404 in the workpiece 1322 after the hole 1404 is suspected to be of inadequate quality and flag the actual location 1348 of the hole 1404 in the workpiece 1322 after the hole 1404 is suspected to be of inadequate quality for confirmation of the inadequate quality and further disposition.

In still yet another further example, the at least one processor 1306 and the associated memory 1308, in conjunction with the at least one processor 1306 running the drilling operation analysis application program 1314, are configured to verify the stiffness value 1332 predicted by the neural network 1316 is within an acceptable tolerance 1350 of an expected stiffness value 1346. In an even further example, the at least one processor 1306 and the associated memory 1308, in conjunction with the at least one processor 1306 running the drilling operation analysis application program 1314, are configured to send the one or more displacement feature 1362 and the stiffness value 1332 to the neural network 1316 as supplemental training data to implement machine learning for the neural network 1316. In another even further example, the at least one processor 1306 and the associated memory 1308, in conjunction with the at least one processor 1306 running the drilling operation analysis application program 1314, are configured to send the one or more displacement feature 1362 and the stiffness value 1332 predicted by the neural network 1316 to a training data repository 1352 as supplemental training data for use during a training mode of the neural network 1316.

In another further example, the neural network 1316 is configured to predict a drilling location 1354 of the hole 1404 through the workpiece 1322 in relation to a preceding hole 1408 with a tack fastener 1406 installed therein based on a regression analysis of the one or more displacement feature 1362 by the neural network 1316 during the processing of the one or more displacement feature 1362. In an even further example, the at least one processor 1306 and the associated memory 1308, in conjunction with the at least one processor 1306 running the drilling operation analysis application program 1314, are configured to compare the drilling location 1354 predicted by the neural network 1316 to a predetermined threshold 1342 for an expected drilling location 1356 of the hole 1404, determine the preceding hole 1408 in the workpiece 1322 is suspected to be missing the tack fastener 1406 where the drilling location 1354 predicted by the neural network 1316 is greater than the predetermined threshold 1342, identify an actual location 1348 of the hole 1404 in the workpiece 1322 after the tack fastener 1406 is suspected to be missing and flag the actual location 1348 of the hole 1404 in the workpiece 1322 after the tack fastener 1406 is suspected to be missing for confirmation the tack fastener 1406 is missing and further disposition.

In another even further example, the at least one processor 1306 and the associated memory 1308, in conjunction with the at least one processor 1306 running the drilling operation analysis application program 1314, are configured to verify the drilling location 1354 predicted by the neural network 1316 is within an acceptable tolerance 1350 of an expected drilling location 1356. In an even yet further example, the at least one processor 1306 and the associated memory 1308, in conjunction with the at least one processor 1306 running the drilling operation analysis application program 1314, are configured to send the one or more displacement feature 1362 and the drilling location 1354 to the neural network 1316 as supplemental training data to implement machine learning for the neural network 1316. In another even yet further example, the at least one processor 1306 and the associated memory 1308, in conjunction with the at least one processor 1306 running the drilling operation analysis application program 1314, are configured to send the one or more displacement feature 1362 and the drilling location 1354 predicted by the neural network 1316 to a training data repository 1352 as supplemental training data for use during a training mode of the neural network 1316.

In addition to components disclosed above, in yet another example, the analytical computing device 1300 also includes a user input device 1366 and a user display device 1368.

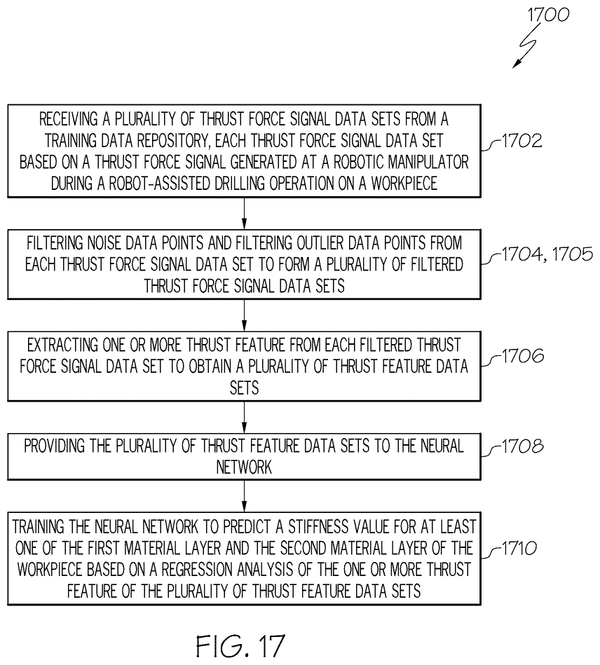

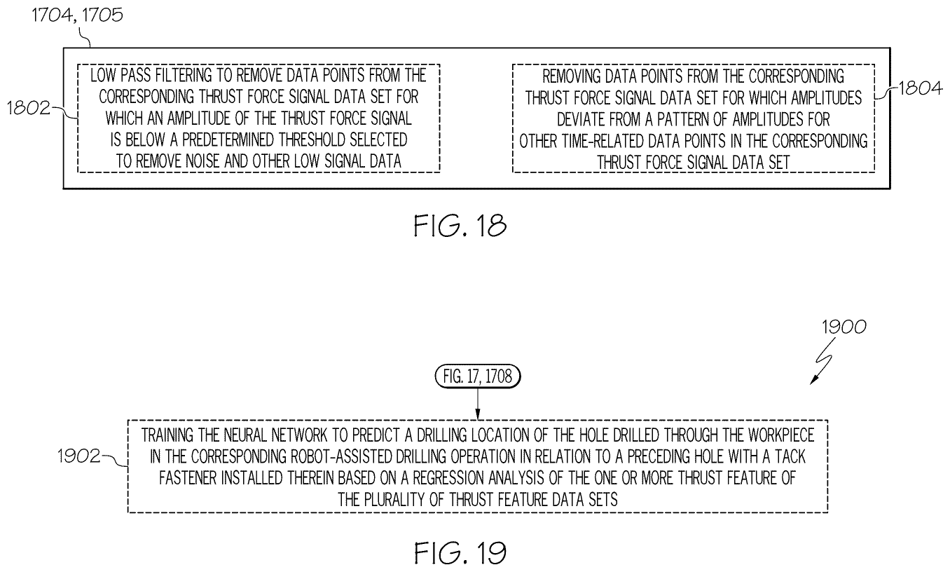

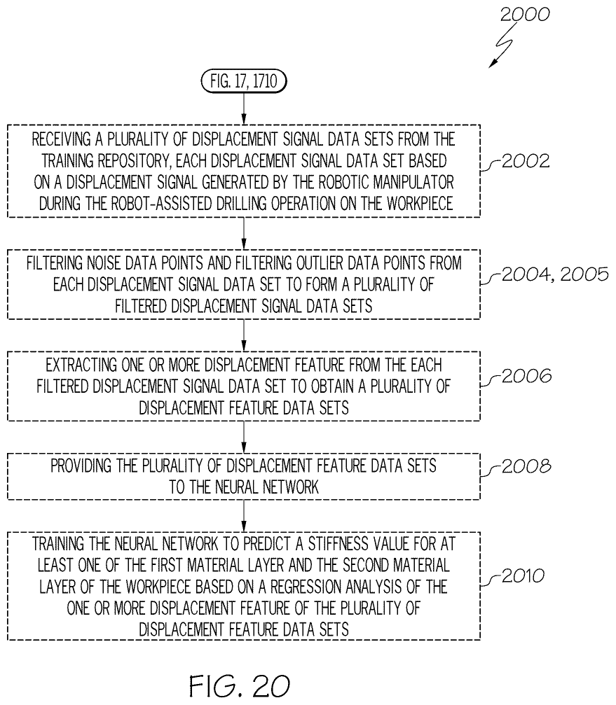

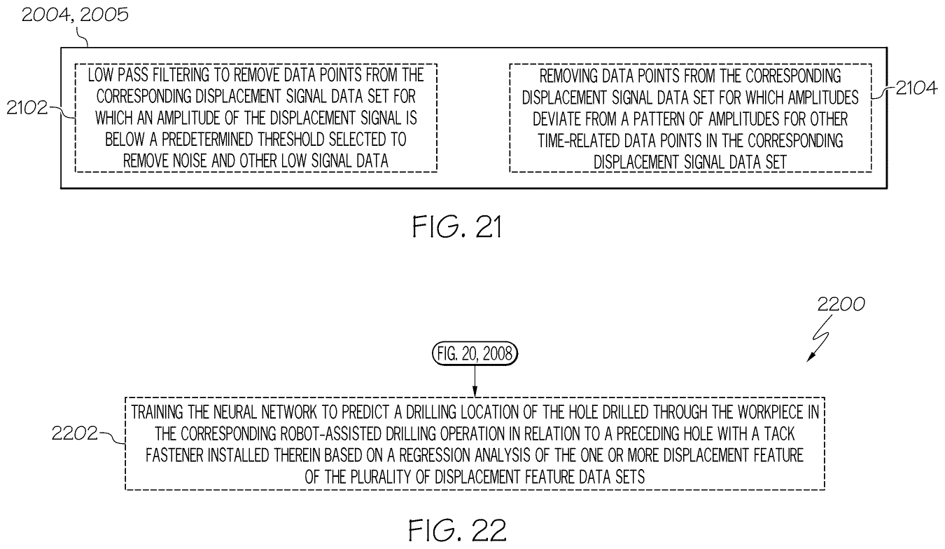

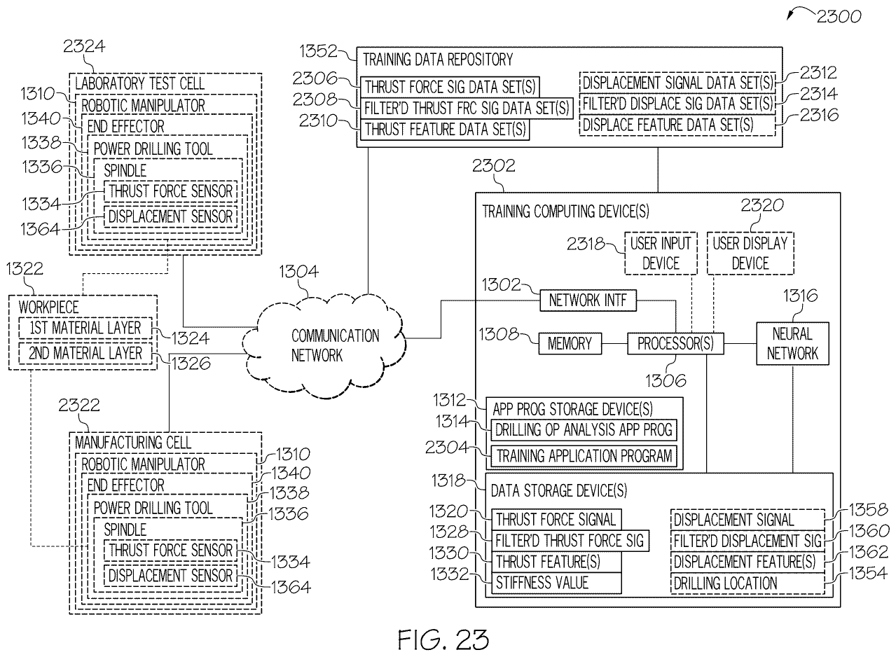

Referring generally to FIGS. 13-23, by way of examples, the present disclosure is directed to methods 1700, 1900, 2000, 2200 for training neural networks 1316 to analyze robot-assisted drilling operations 1402. FIG. 13 is a functional block diagram of an example of an analytical computing device 1300. FIG. 14 is a functional perspective drawing of a robotic manipulator 1310 monitoring fabrication of a workpiece 1322 during a drilling operation 1402. FIG. 15 is a graph 1500 of a thrust force signal 1320 over time during a robot-assisted drilling operation 1402 on a workpiece 1322. FIG. 16 is a graph 1600 of a displacement signal 1358 over time during a robot-assisted drilling operation 1402 on a workpiece 1322. FIG. 17 provides an example of the method 1700 for training a neural network 1316 to analyze a robot-assisted drilling operation 1402. FIG. 18 provides an example the filtering 1704 of noise data points and the filtering 1705 of outlier data points from FIG. 17. FIG. 19, in combination with FIG. 17, provides an example of the method 1900 for training a neural network 1316 to analyze a robot-assisted drilling operation 1402. FIG. 20, in combination with FIG. 17, provides an example of the method 2000 for training a neural network 1316 to analyze a robot-assisted drilling operation 1402. FIG. 21 provides an example the filtering 2004 of noise data points and the filtering 2005 of outlier data points from FIG. 20. FIG. 22, in combination with FIGS. 17 and 20, provides an example of the method 2200 for training a neural network 1316 to analyze a robot-assisted drilling operation 1402. FIG. 23 is a functional block diagram of an example of a training system 2300.

With reference again to FIGS. 13-15, 17, 18 and 23, in one or more example, a method 1700 (see FIG. 17) for training a neural network 1316 to analyze a robot-assisted drilling operation 1402 includes receiving 1702 a plurality of thrust force signal data sets 2306 from a training data repository 1352. Each thrust force signal data set 2306 based on a thrust force signal 1320 generated at a robotic manipulator 1310 during a corresponding robot-assisted drilling operation 1402 on a workpiece 1322. The workpiece 1322 includes a first material layer 1324 and a second material layer 1326. The robot-assisted drilling operation 1402 includes drilling a hole 1404 for a fastener through the first material layer 1324 and then through the second material layer 1326. At 1704, 1705, noise data points 1504 are filtered and outlier data points 1506 are filtered from each thrust force signal data set 2306 to form a plurality of filtered thrust force signal data sets 2308. At 1706, one or more thrust feature 1330 is extracted from each filtered thrust force signal data set 2308 to obtain a plurality of thrust feature data sets 2310. Each thrust feature data set 2310 including the one or more thrust feature 1330. At 1708, the plurality of thrust feature data sets 2310 are provided to the neural network 1316. At 1710, the neural network 1316 is trained to predict a stiffness value 1332 for at least one of the first material layer 1324 and the second material layer 1326 of the workpiece 1322 based on a regression analysis of the one or more thrust feature 1330 of the plurality of thrust feature data sets 2310.

In another example of the method 1700, the thrust force signal 1320 is generated by a thrust force sensor 1334 associated with a spindle 1336 of a power drilling tool 1338 on an end effector 1340 of the robotic manipulator 1310 during the robot-assisted drilling operation 1402. In yet another example of the method 1700, the thrust force signal 1320 is indicative of a thrust force exerted upon the workpiece 1322 by the robotic manipulator 1310 over time during the robot-assisted drilling operation 1402. In still another example of the method 1700, the filtering 1704 of the noise data points 1504 includes low pass filtering 1802 to remove data points from the corresponding thrust force signal data set 2306 for which an amplitude of the thrust force signal 1320 is below a predetermined threshold 1342 selected to remove noise and other low signal data. In still yet another example of the method 1700, the filtering 1705 of the outlier data points 1506 includes removing 1804 data points from the corresponding thrust force signal data set 2306 for which amplitudes deviate from a pattern 1344 of amplitudes for other time-related data points in the corresponding thrust force signal data set 2306. In another example of the method 1700, the one or more thrust feature 1330 includes a width of each peck 1508 during the robot-assisted drilling operation 1402, an amount of pecks 1508 required to complete the robot-assisted drilling operation 1402, a thrust signal amplitude delta for each peck 1508 during the robot-assisted drilling operation 1402 or any other suitable thrust feature in any suitable combination. In yet another example of the method 1700, the neural network 1316 includes a multi-layer perceptron model, a convolutional model, a recurrent model, a regression model, a classification model, a feedforward model, a back propagation model or any other suitable neural network model in any suitable combination.

With reference again to FIGS. 13, 14, 17, 19 and 23, in one or more example, a method 1900 (see FIG. 19) for training a neural network 1316 to analyze a robot-assisted drilling operation 1402 includes the method 1700 of FIG. 17 and continues from 1708 to 1902 where the neural network 1316 is trained to predict a drilling location 1354 of the hole 1404 drilled through the workpiece 1322 in the corresponding robot-assisted drilling operation 1402 in relation to a preceding hole 1408 with a tack fastener 1406 installed therein based on a regression analysis of the one or more thrust feature 1330 of the plurality of thrust feature data sets 2310.

With reference again to FIGS. 13, 14, 16, 17, 20, 21 and 23, in one or more example, a method 2000 (see FIG. 20) for training a neural network 1316 to analyze a robot-assisted drilling operation 1402 includes the method 1700 of FIG. 17 and continues from 1710 to 2002 where a plurality of displacement signal data sets 2312 are received from the training data repository 1352. Each displacement signal data set 2312 based on a displacement signal 1358 generated by the robotic manipulator 1310 during the corresponding robot-assisted drilling operation 1402 on the workpiece 1322. At 2004, 2005, noise data points 1604 are filtered and outlier data points 1606 are filtered from each displacement signal data set 2312 to form a plurality of filtered displacement signal data sets 2314. At 2006, one or more displacement feature 1362 is extracted from each filtered displacement signal data set 2314 to obtain a plurality of displacement feature data sets 2316. Each displacement feature data set 2316 including the one or more displacement feature 1362. At 2008, the plurality of displacement feature data sets 2316 are provided to the neural network 1316. At 2010, the neural network 1316 is trained to predict a stiffness value 1332 for at least one of the first material layer 1324 and the second material layer 1326 of the workpiece 1322 based on a regression analysis of the one or more displacement feature of the plurality of displacement feature data sets 2316.

In a further example, the displacement signal 1358 is generated by a displacement sensor 1364 associated with a spindle 1336 of a power drilling tool 1338 on an end effector 1340 of the robotic manipulator 1310 during the robot-assisted drilling operation 1402. In an even further example, the displacement signal 1358 is indicative of movement of the power drilling tool 1338 toward the workpiece 1322 during the robot-assisted drilling operation 1402. In another further example, the filtering 2004 of the noise data points 1604 includes low pass filtering 2102 to remove data points from the corresponding displacement signal data set 2312 for which an amplitude of the displacement signal 1358 is below a predetermined threshold 1342 selected to remove noise and other low signal data. In yet another further example, the filtering 2005 of the outlier data points 1606 includes removing 2104 data points from the corresponding displacement signal data set 2312 for which amplitudes deviate from a pattern 1344 of amplitudes for other time-related data points in the corresponding displacement signal data set 2312. In still another further example, the one or more displacement feature 1362 includes a width of each peck 1608 during the robot-assisted drilling operation 1402, an amount of pecks 1608 required to complete the robot-assisted drilling operation 1402 and a displacement amplitude delta for each peck 1608 during the robot-assisted drilling operation 1402.

With reference again to FIGS. 13, 14, 16, 17, 20, 22 and 23, in one or more example, a method 2200 (see FIG. 22) for training a neural network 1316 to analyze a robot-assisted drilling operation 1402 includes the method 1700 of FIG. 17, the method 2000 of FIG. 20 and continues from 2008 to 2202 where the neural network 1316 is trained to predict a drilling location 1354 of the hole 1404 drilled through the workpiece 1322 in the corresponding robot-assisted drilling operation 1402 in relation to a preceding hole 1408 with a tack fastener 1406 installed therein based on a regression analysis of the one or more displacement feature 1362 of the plurality of displacement feature data sets 2316.

Referring generally to FIG. 23, by way of examples, the present disclosure is directed to training systems 2300 for training neural networks 1316 to analyze robot-assisted drilling operations 1402. FIG. 23 is a functional block diagram of an example of a training system 2300. The training system 2300 includes a training data repository 1352 and a training computing device 2302 in operative communication with the training data repository 1352. In addition to components disclosed above, in another example, the training computing device 2302 also includes a user input device 2318 and a user display device 2320. The training data repository 1352 in operative communication with a manufacturing cell 2322 and a laboratory test cell 2324 via the communication network 1304. In one example, the manufacturing cell 2322 includes the robotic manipulator 1310. In another example, the laboratory test cell 2324 includes the robotic manipulator 1310.

Examples of the methods 100, 300, 400, 500, 600, 700, 900, 1000, 1100, 1200 for monitoring fabrication of workpieces 1322, analytical computing devices 1300, and methods 1700, 1900, 2000, 2200 for training neural networks 1316 to analyze robot-assisted drilling operations 1402 may be related to or used in the context of aircraft manufacturing. Although an aircraft example is described, the examples and principles disclosed herein may be applied to other products in the aerospace industry and other industries, such as the automotive industry, the space industry, the construction industry and other design and manufacturing industries. Accordingly, in addition to aircraft, the examples and principles disclosed herein may apply to the use of various products in the manufacture of various types of vehicles and in the construction of various types of buildings.

The preceding detailed description refers to the accompanying drawings, which illustrate specific examples described by the present disclosure. Other examples having different structures and operations do not depart from the scope of the present disclosure. Like reference numerals may refer to the same feature, element, or component in the different drawings. Throughout the present disclosure, any one of a plurality of items may be referred to individually as the item and a plurality of items may be referred to collectively as the items and may be referred to with like reference numerals. Moreover, as used herein, a feature, element, component, or step preceded with the word “a” or “an” should be understood as not excluding a plurality of features, elements, components, or steps, unless such exclusion is explicitly recited.

Illustrative, non-exhaustive examples, which may be, but are not necessarily, claimed, of the subject matter according to the present disclosure are provided above. Reference herein to “example” means that one or more feature, structure, element, component, characteristic and/or operational step described in connection with the example is included in at least one aspect, embodiment and/or implementation of the subject matter according to the present disclosure. Thus, the phrases “an example,” “another example,” “one or more example,” and similar language throughout the present disclosure may, but do not necessarily, refer to the same example. Further, the subject matter characterizing any one example may, but does not necessarily, include the subject matter characterizing any other example. Moreover, the subject matter characterizing any one example may be, but is not necessarily, combined with the subject matter characterizing any other example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, device, structure, article, element, component, or hardware “configured to” perform a specified function is specifically selected, created, implemented, utilized, programmed and/or designed for the purpose of performing the specified function. As used herein, “configured to” denotes existing characteristics of a system, apparatus, structure, article, element, component, or hardware that enable the system, apparatus, structure, article, element, component, or hardware to perform the specified function without further modification. For purposes of this disclosure, a system, apparatus, device, structure, article, element, component, or hardware described as being “configured to” perform a particular function may additionally or alternatively be described as being “adapted to” and/or as being “operative to” perform that function.

Unless otherwise indicated, the terms “first,” “second,” “third,” etc., are used herein merely as labels and are not intended to impose ordinal, positional, or hierarchical requirements on the items to which these terms refer. Moreover, reference to, e.g., a “second” item does not require or preclude the existence of, e.g., a “first” or lower-numbered item and/or, e.g., a “third” or higher-numbered item.

As used herein, the phrase “at least one of,” when used with a list of items, means different combinations of one or more of the listed items may be used and only one of each item in the list may be needed. For example, “at least one of item A, item B and item C” may include, without limitation, item A or item A and item B. This example also may include item A, item B and item C or item B and item C. In other examples, “at least one of” may be, for example, without limitation, two of item A, one of item B and ten of item C; four of item B and seven of item C; and other suitable combinations. As used herein, the term “and/or” and the “/” symbol includes any and all combinations of one or more of the associated listed items.

As used herein, the terms “coupled,” “coupling,” and similar terms refer to two or more elements that are joined, linked, fastened, attached, connected, put in communication, or otherwise associated (e.g., mechanically, electrically, fluidly, optically, electromagnetically) with one another. In various examples, the elements may be associated directly or indirectly. As an example, element A may be directly associated with element B. As another example, element A may be indirectly associated with element B, for example, via another element C. It will be understood that not all associations among the various disclosed elements are necessarily represented. Accordingly, couplings other than those depicted in the figures may also exist.

As used herein, the term “approximately” refers to or represents a condition that is close to, but not exactly, the stated condition that still performs the desired function or achieves the desired result. As an example, the term “approximately” refers to a condition that is within an acceptable predetermined tolerance or accuracy, such as to a condition that is within 10% of the stated condition. However, the term “approximately” does not exclude a condition that is exactly the stated condition. As used herein, the term “substantially” refers to a condition that is essentially the stated condition that performs the desired function or achieves the desired result.

In FIGS. 1-12 and 17-22, referred to above, may represent functional elements, features, or components thereof and do not necessarily imply any particular structure. Accordingly, modifications, additions and/or omissions may be made to the illustrated structure. Additionally, those skilled in the art will appreciate that not all elements, features and/or components described and illustrated in FIGS. 1-12 and 17-22, referred to above, need be included in every example and not all elements, features and/or components described herein are necessarily depicted in each illustrative example. Accordingly, some of the elements, features and/or components described and illustrated in FIGS. 1-12 and 17-22 may be combined in various ways without the need to include other features described and illustrated in FIGS. 1-12 and 17-22, other drawing figures and/or the accompanying disclosure, even though such combination or combinations are not explicitly illustrated herein. Similarly, additional features not limited to the examples presented, may be combined with some or all the features shown and described herein. Unless otherwise explicitly stated, the schematic illustrations of the examples depicted in FIGS. 1-12 and 17-22, referred to above, are not meant to imply structural limitations with respect to the illustrative example. Rather, although one illustrative structure is indicated, it is to be understood that the structure may be modified when appropriate. Accordingly, modifications, additions and/or omissions may be made to the illustrated structure. Furthermore, elements, features and/or components that serve a similar, or at least substantially similar, purpose are labeled with like numbers in each of FIGS. 1-12 and 17-22 and such elements, features and/or components may not be discussed in detail herein with reference to each of FIGS. 1-12 and 17-22. Similarly, all elements, features and/or components may not be labeled in each of FIGS. 1-12 and 17-22, but reference numerals associated therewith may be utilized herein for consistency.

In FIGS. 13-16 and 23, referred to above, the blocks may represent operations, steps and/or portions thereof, and lines connecting the various blocks do not imply any particular order or dependency of the operations or portions thereof. It will be understood that not all dependencies among the various disclosed operations are necessarily represented. FIGS. 13-16 and 23 and the accompanying disclosure describing the operations of the disclosed methods set forth herein should not be interpreted as necessarily determining a sequence in which the operations are to be performed. Rather, although one illustrative order is indicated, it is to be understood that the sequence of the operations may be modified when appropriate. Accordingly, modifications, additions and/or omissions may be made to the operations illustrated and certain operations may be performed in a different order or simultaneously. Additionally, those skilled in the art will appreciate that not all operations described need be performed.

Further, references throughout the present specification to features, advantages, or similar language used herein do not imply that all the features and advantages that may be realized with the examples disclosed herein should be, or are in, any single example. Rather, language referring to the features and advantages is understood to mean that a specific feature, advantage, or characteristic described in connection with an example is included in at least one example. Thus, discussion of features, advantages and similar language used throughout the present disclosure may, but does not necessarily, refer to the same example.

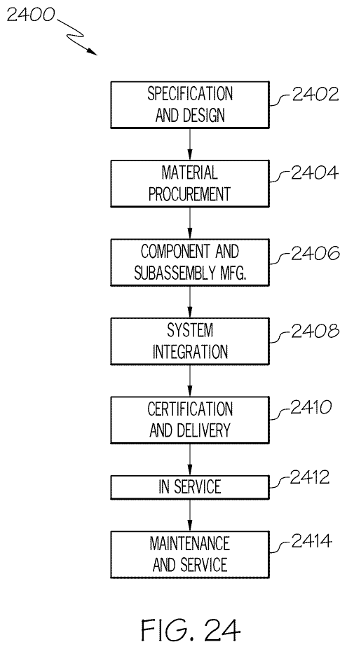

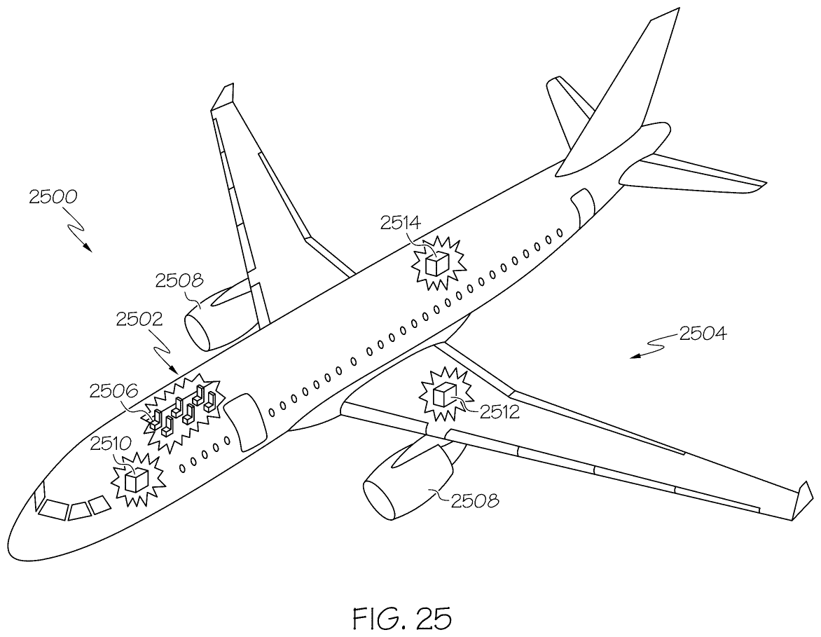

Examples of the subject matter disclosed herein may be described in the context of aircraft manufacturing and service method 2400 as shown in FIG. 24 and aircraft 2500 as shown in FIG. 25. In one or more example, the disclosed methods 100, 300, 400, 500, 600, 700, 900, 1000, 1100, 1200 for monitoring fabrication of workpieces 1322, analytical computing devices 1300, and methods 1700, 1900, 2000, 2200 for training neural networks 1316 to analyze robot-assisted drilling operations 1402 may be used in aircraft manufacturing. During pre-production, the service method 2400 may include specification and design (block 2402) of aircraft 2500 and material procurement (block 2404). During production, component and subassembly manufacturing (block 2406) and system integration (block 2408) of aircraft 2500 may take place. Thereafter, aircraft 2500 may go through certification and delivery (block 2410) to be placed in service (block 2412). While in service, aircraft 2500 may be scheduled for routine maintenance and service (block 2414). Routine maintenance and service may include modification, reconfiguration, refurbishment, etc. of one or more systems of aircraft 2500.

Each of the processes of the service method 2400 may be performed or carried out by a system integrator, a third party and/or an operator (e.g., a customer). For the purposes of this description, a system integrator may include, without limitation, any number of aircraft manufacturers and major-system subcontractors; a third party may include, without limitation, any number of vendors, subcontractors and suppliers; and an operator may be an airline, leasing company, military entity, service organization and so on.

As shown in FIG. 25, aircraft 2500 produced by the service method 2400 may include airframe 2502 with a plurality of high-level systems 2504 and interior 2506. Examples of high-level systems 2504 include one or more of propulsion system 2508, electrical system 2810, hydraulic system 2812 and environmental system 2814. Any number of other systems may be included. Although an aerospace example is shown, the principles disclosed herein may be applied to other industries, such as the automotive industry. Accordingly, in addition to aircraft 2500, the principles disclosed herein may apply to other vehicles, e.g., land vehicles, marine vehicles, space vehicles, etc.

The disclosed the methods 100, 300, 400, 500, 600, 700, 900, 1000, 1100, 1200 for monitoring fabrication of workpieces 1322, analytical computing devices 1300, and methods 1700, 1900, 2000, 2200 for training neural networks 1316 to analyze robot-assisted drilling operations 1402 For example, components or subassemblies corresponding to component and subassembly manufacturing (block 2406) may be fabricated or manufactured in a manner similar to components or subassemblies produced while aircraft 2500 is in service (block 2412). Also, one or more example of the system(s), method(s), or combination thereof may be utilized during production stages (block 2406 and block 2408), for example, by substantially expediting assembly of or reducing the cost of aircraft 2500. Similarly, one or more example of the system or method realizations, or a combination thereof, may be utilized, for example and without limitation, while aircraft 2500 is in service (block 2412) and/or during maintenance and service (block 2414).

The described features, advantages and characteristics of one example may be combined in any suitable manner in one or more other examples. One skilled in the relevant art will recognize that the examples described herein may be practiced without one or more of the specific features or advantages of a particular example. In other instances, additional features and advantages may be recognized in certain examples that may not be present in all examples. Furthermore, various examples of the methods 100, 300, 400, 500, 600, 700, 900, 1000, 1100, 1200 for monitoring fabrication of workpieces 1322, analytical computing devices 1300, and methods 1700, 1900, 2000, 2200 for training neural networks 1316 to analyze robot-assisted drilling operations 1402 have been shown and described, modifications may occur to those skilled in the art upon reading the specification. The present application includes such modifications and is limited only by the scope of the claims.

1. A method for monitoring fabrication of a workpiece, comprising:
receiving a thrust force signal from a robotic manipulator during a robot-assisted drilling operation on the workpiece, the workpiece comprising a first material layer and a second material layer, the robot-assisted drilling operation comprising drilling a hole for a fastener through the first material layer and then through the second material layer;
filtering noise data points and filtering outlier data points from the thrust force signal to form a filtered thrust force signal;
extracting one or more thrust feature from the filtered thrust force signal;
processing the one or more thrust feature using a neural network trained to analyze the robot-assisted drilling operation; and
predicting a stiffness value for at least one of the first material layer and the second material layer of the workpiece based on a regression analysis of the one or more thrust feature by the neural network during the processing of the one or more thrust feature.

2. The method of claim 1 wherein the thrust force signal is generated by a thrust force sensor associated with a spindle of a power drilling tool on an end effector of the robotic manipulator during the robot-assisted drilling operation.

3. The method of claim 1 wherein the thrust force signal is indicative of a thrust force exerted upon the workpiece by the robotic manipulator over time during the robot-assisted drilling operation.

4. The method of claim 1 wherein the robotic manipulator comprises at least one of a robotic arm, an articulated robotic arm, a 4-axis robotic arm, a 6-axis robotic arm, a 7-axis robotic arm and a collaborative robot.

5. The method of claim 1 wherein the first material layer of the workpiece comprises at least one of a polymeric composite material, a thermoplastic composite material, a thermoset composite material, a metallic material, an aluminum material, an aluminum alloy material, a titanium material and a titanium alloy material.

6. The method of claim 1 wherein the second material layer of the workpiece comprises at least one of a metallic material, an aluminum material, an aluminum alloy material, a titanium material, a titanium alloy material, a polymeric composite material, a thermoplastic composite material and a thermoset composite material.

7. The method of claim 1 wherein the robot-assisted drilling operation further comprises installing a tack fastener in the hole through the first material layer and the second material layer.

8. The method of claim 1, the filtering of the noise data points comprising:
low pass filtering to remove data points in which an amplitude of the thrust force signal is below a predetermined threshold selected to remove noise and other low signal data from the thrust force signal.

9. The method of claim 1, the filtering of the outlier data points comprising:
removing data points with amplitudes that deviate from a pattern of amplitudes for other time-related data points.

10. The method of claim 1 wherein the one or more thrust feature comprises at least one of a width of each peck during the robot-assisted drilling operation, an amount of pecks required to complete the robot-assisted drilling operation and a thrust signal amplitude delta for each peck during the robot-assisted drilling operation.

11. The method of claim 1 wherein the neural network comprises at least one of a multi-layer perceptron model, a convolutional model, a recurrent model, a regression model, a classification model, a feedforward model and a back propagation model.

12. The method of claim 1, further comprising:
comparing the stiffness value from the predicting to a predetermined threshold for an expected stiffness value for the workpiece;
determining the hole in the workpiece is suspected to be of inadequate quality where the stiffness value from the predicting is less than the predetermined threshold;
identifying an actual location of the hole in the workpiece after the hole is suspected to be of inadequate quality; and
flagging the actual location of the hole in the workpiece after the hole is suspected to be of inadequate quality for confirmation of the inadequate quality and further disposition.

13. The method of claim 1, further comprising:
verifying the stiffness value from the predicting is within an acceptable tolerance of an expected stiffness value.

14-15. (canceled)

16. The method of claim 1, further comprising:
predicting a drilling location of the hole through the workpiece in relation to a preceding hole with a tack fastener installed therein based on a regression analysis of the one or more thrust feature by the neural network during the processing of the one or more thrust feature.

17. The method of claim 16, further comprising:
comparing the drilling location from the predicting to a predetermined threshold for an expected drilling location of the hole;
determining the preceding hole in the workpiece is suspected to be missing the tack fastener where the drilling location from the predicting is greater than the predetermined threshold;
identifying an actual location of the hole in the workpiece after the tack fastener is suspected to be missing; and
flagging the actual location of the hole in the workpiece after the tack fastener is suspected to be missing for confirmation the tack fastener is missing and further disposition.

18. The method of claim 16, further comprising:
verifying the drilling location from the predicting is within an acceptable tolerance of an expected drilling location.

19-20. (canceled)

21. The method of claim 1, further comprising:
receiving a displacement signal from the robotic manipulator during the robot-assisted drilling operation on the workpiece;
filtering noise data points and filtering outlier data points from the displacement signal to form a filtered displacement signal;
extracting one or more displacement feature from the filtered displacement signal;
processing the one or more displacement feature using the neural network trained to analyze the robot-assisted drilling operation; and
predicting a stiffness value for at least one of the first material layer and the second material layer of the workpiece based on a regression analysis of the one or more displacement feature by the neural network during the processing of the one or more displacement feature.

22. The method of claim 21 wherein the displacement signal is generated by a displacement sensor associated with a spindle of a power drilling tool on an end effector of the robotic manipulator during the robot-assisted drilling operation.

23-35. (canceled)

36. An analytical computing device, comprising:
a network interface in operative communication with a communication network;
at least one processor and associated memory; the at least one processor in operative communication with a robotic manipulator via the network interface and the communication network;
at least one application program storage device storing a drilling operation analysis application program;
a neural network trained to analyze a data set associated with a robot-assisted drilling operation; and
at least one data storage device configured to at least temporarily store the data set associated with the robot-assisted drilling operation;
wherein the at least one processor and the network interface are configured to receive a thrust force signal from the robotic manipulator via the communication network during the robot-assisted drilling operation on a workpiece, the workpiece comprising a first material layer and a second material layer, the robot-assisted drilling operation comprising drilling a hole for a fastener through the first material layer and then through the second material layer;
wherein the at least one processor and the associated memory, in conjunction with the at least one processor running the drilling operation analysis application program, are configured to filter noise data points and outlier data points from the thrust force signal to form a filtered thrust force signal, extract one or more thrust feature from the filtered thrust force signal and provide the one or more thrust feature to the neural network; and
wherein the neural network is configured to process the one or more thrust feature, perform a regression analysis on the one or more thrust feature, predict a stiffness value for at least one of the first material layer and the second material layer of the workpiece based at least in part on the regression analysis and at least temporarily store the stiffness value in the at least one data storage device.

37-64. (canceled)

65. A method for training a neural network to analyze a robot-assisted drilling operation, comprising:
receiving a plurality of thrust force signal data sets from a training data repository, each thrust force signal data set based on a thrust force signal generated at a robotic manipulator during a corresponding robot-assisted drilling operation on a workpiece, the workpiece comprising a first material layer and a second material layer, the robot-assisted drilling operation comprising drilling a hole for a fastener through the first material layer and then through the second material layer;
filtering noise data points and filtering outlier data points from each thrust force signal data set to form a plurality of filtered thrust force signal data sets;
extracting one or more thrust feature from each filtered thrust force signal data set to obtain a plurality of thrust feature data sets, each thrust feature data set comprising the one or more thrust feature;
providing the plurality of thrust feature data sets to the neural network; and
training the neural network to predict a stiffness value for at least one of the first material layer and the second material layer of the workpiece based on a regression analysis of the one or more thrust feature of the plurality of thrust feature data sets.

66-79. (canceled)
